(19) United States
(12) Patent Application Publication
(10) Pub. No.: US 2004/0173501 A1
(43) Pub. Date: Sep. 9, 2004

(54) Methods for treating organic compounds and treated organic compounds

(75) Inventors: Keith H. Lawson, Ponca City, OK (US); Douglas S. Jack, Ponca City, OK (US); Craig T. Barker, Ponca City, OK (US); Sridhar Gopalakrishnan, Ponca City, OK (US); Rafael L. Espinoza, Ponca City, OK (US)

Correspondence Address: DAVID W. WESTPHAL, CONOCOPHILLIPS COMPANY, P.O. BOX 1267, PONCA CITY, OK, 74602-1267, US

(73) Assignee: ConocoPhillips Company, Houston, TX (US)

(21) Appl. No.: 10/382,339

(22) Filed: Mar. 5, 2003

(51) Int. Cl. C10L 1/08; C10G 45/00; C07C 1/00; C10G 65/12

(52) U.S. Cl. 208/15; 208/144; 208/108; 208/89; 585/258; 518/715; 518/726

(57) ABSTRACT

Embodiments include processes for producing streams containing organic molecules (for example, diesel fuels and diesel fuel blending agents) including ultra-low severity hydrotreatment of at least a portion of a hydrocarbon synthesis product stream. Also, streams containing organic molecules (for example, diesel fuels and diesel fuel blending agents) produced by the processes are described.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

The present invention relates to methods and processes comprising a ultra-low severity hydrotreatment of a hydrocarbon stream for producing middle distillates. Particularly the present invention relates to processes comprising a ultra-low severity hydrotreatment of a hydrocarbon stream derived from synthesis gas.

### BACKGROUND OF THE INVENTION

Natural gas is a naturally-occurring abundant energy resource. Wells that provide natural gas are often remote from locations with a demand for its consumption. The costs associated with transporting natural gas from these remote wells are generally very high and may not be economical.

As opposed to natural gas, fuels that are liquid under standard atmospheric conditions have the advantage that they can be transported more economically. Thus, there has been interest in developing technologies for converting natural gas to more readily transportable liquid fuels.

One method for converting natural gas to liquid fuels involves two sequential chemical transformations. In the first transformation natural gas (which contains primarily methane) is reacted to form a mixture of CO and H₂ (“synthesis gas” or “syngas”). This syngas generation usually occurs by dry reforming, steam reforming, or partial oxidation, respective examples of which are set forth below for methane:

CH₄+CO₂→2CO+2H₂ (1)

CH₄+H₂O→CO+3H₂ (2)

CH₄+½O₂→CO+2H₂ (3)

Examples of syngas generation processes are disclosed in U.S. Pat. No. 6,402,989 to Gaffney and Gunardson, Harold, “Industrial Gases in Petrochemical Processing” 41-80 (1998), both incorporated herein by reference.

In the second transformation, hydrocarbon synthesis, (by way of example only, the Fischer-Tropsch process), carbon monoxide reacts with hydrogen to form organic molecules. An example of a Fischer-Tropsch process is disclosed in U.S. Pat. No. 6,333,294 to Chao et al., incorporated herein by reference. The product stream produced by conversion of natural gas commonly contains a range of hydrocarbons including light gases, gases, light naphtha, naphtha, kerosene, diesel, heavy diesel, heavy oil's, waxes, and heavy waxes. These cuts are approximate and there is some degree of overlapping of components in each range. The product stream also often contains many byproducts such as olefins (i.e., hydrocarbons containing at least one carbon-carbon double bond) and heteroatomic compounds (e.g., aldehydes, alcohols).

Usually, the most valuable fractions of a hydrocarbon synthesis product stream are the middle distillate fractions. The middle distillates or “middle cuts” generally comprise kerosene, diesel, heating oil, heavy diesel, and heavy oils. Thus, it is desirable to maximize the production of the middle distillates. One method for increasing the production of middle distillates is to crack the heavy waxy products to middle distillate range molecules. For example, a method of processing syncrude to produce diesel fuel may include distillation to separate diesel and wax fractions from the lighter fraction, cracking of the wax fraction, and further distillation of the cracked product to separate its diesel fraction. The diesel fraction is then often blended with other compounds to produce commercial diesel products. It is within the skill of one of ordinary skill in the art to determine which products are desirable based on the intended uses of the products. Likewise, separation of the components of a hydrocarbon stream is well known in the art, and it will be within the ability of one of ordinary skill in the art to determine the conditions which will effect the desired separation. In the instance of a distillation column, it will be within the ability of one of ordinary skill in the art to determine various parameters such as height and internal design of the column, the distillation temperature, the feed location, and the product withdrawal/sidedraw locations.

Another traditional step in the preparation of products derived from a hydrocarbon synthesis product stream (such as from a Fischer-Tropsch synthesis) is hydrotreatment. Traditionally, hydrotreatment takes place at temperatures of at least 350° F. and usually from about 380° F. to about 450° F. over a nickel catalyst. Under these conditions, traditional hydrotreatment removes olefins that are known to cause chemical instability. This instability frequently manifests itself in the formation of gums which may form solid deposits in the fuel system and engine. This instability is typically measured by the oxidation stability ASTM D2274 test. Traditional hydrotreatment also removes heteroatomic compounds such as sulfur-containing compounds, oxygenates and amines.

Specific examples of methods of processing a Fischer-Tropsch product stream are disclosed in various patents: U.S. Pat. Nos. 6,296,757; 5,766,274; 5,378,348 and patent applications WO 00/20535; WO 01/59034. While some methods describe the use of a hydrotreatment step above 350° F., more typically around 400° F.-450° F. with a nickel catalyst, others do not hydrotreat certain cuts of the Fischer-Tropsch product stream in order to retain some oxygenates into the final product. Oxygenates indeed have been shown to be beneficial in the Fischer-Tropsch products as disclosed in U.S. Pat. No. 5,645,613.

Although it is desirable to hydrotreat at least a portion of the hydrocarbon synthesis products to remove olefins (which render the product (e.g., diesel) unstable), the disclosed hydrotreating schemes have the additional effect of also converting the heteroatomic compounds (e.g., oxygenates). When present, the oxygenates (particularly alcohols) may advantageously increase the lubricity of the product. Others have reported methods to maintain the oxygenates in the diesel fraction of a hydrocarbon synthesis product stream by causing the diesel fraction to avoid hydrotreatment.

### SUMMARY

Surprisingly it has been found that ultra-low severity hydrotreating, for example, with a conventional nickel catalyst at 350 psia of hydrogen partial pressure in the hydrotreater outlet, at 250° F. and a liquid hourly space velocity of about 3 hr⁻¹, is likely to cause only partial conversion (i.e., converts substantially all of the olefins while leaving a substantial amount of the heteroatoms comprised in the heteroatomic compounds (e.g., oxygenates) attached to their parent molecules), and that this partial conversion can enhance the lubricity of the diesel fuel.

In accordance with the present invention, there are herein disclosed methods and processes for producing a distillate. Some embodiments disclosed herein comprise a hydrotreater for ultra-low severity hydrotreatment of a hydrocarbon synthesis product stream and removing much of the undesirable byproducts and impurities while leaving at least some of the oxygenates, and a fractionation unit for separating the hydrotreater effluent. Additional process embodiments disclosed herein comprise ultra-low severity hydrotreating of a hydrocarbon synthesis product stream, hydrocracking of a heavy fraction of the hydrocarbon synthesis product stream, and fractionating in order to produce middle distillate.

### BRIEF DESCRIPTION OF THE DRAWINGS

For a more detailed description, reference will now be made to the accompanying figures.

FIG. 1 is a schematic drawing of a first reactor scheme in accordance with an embodiment of the present invention.

FIG. 2 is a schematic drawing of a second reactor scheme in accordance with an embodiment of the present invention.

FIG. 3 is a schematic drawing of a third reactor scheme in accordance with an embodiment of the present invention.

FIG. 4 is a schematic drawing of a fourth reactor scheme in accordance with an embodiment of the present invention.

FIG. 5 is a schematic drawing of a fifth reactor scheme in accordance with an embodiment of the present invention.

FIG. 6 is a schematic drawing of a sixth reactor scheme in accordance with an embodiment of the present invention.

FIG. 7 is a schematic drawing of a seventh reactor scheme in accordance with an embodiment of the present invention.

### DETAILED DESCRIPTION

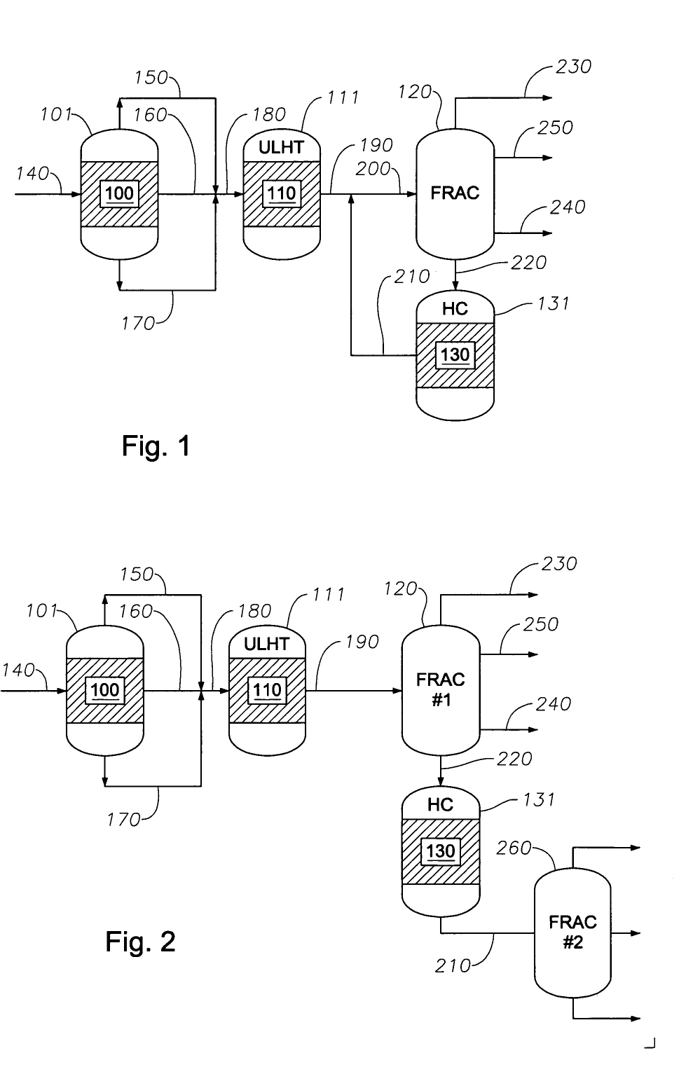

In the reactor scheme of FIG. 1, there is shown a hydrocarbon synthesis reactor 101, an ultra-low severity hydrotreater 111, a fractionation unit 120, and a hydrocracker 131. Feedstream 140 comprising CO and H₂ in preferably about a 2:1H₂:CO molar ratio is fed into hydrocarbon synthesis reactor 101. Reactor 101 includes a hydrocarbon synthesis catalyst in reaction zone 100. Feedstream 140 reacts in reaction zone 100 to produce a product stream 180. Product stream 180 comprises primarily hydrocarbons with 3 carbon atoms or more (C₃₊), preferably hydrocarbons with 5 carbon atoms or more (C₅₊). The product stream 180 is introduced to hydrotreating catalyst 110 in ultra-low severity hydrotreater 111 where stream 180 is hydrotreated. This ultra-low severity hydrotreating saturates the olefinic compounds present in product stream 180 while allowing a substantial amount of the oxygenates to remain unconverted. Advantageously, the ultra-low severity hydrotreater can also remove or reduce solid material that can be present in the product stream 180, particularly when the hydrocarbon synthesis reactor 101 comprises free-flowing or suspended catalyst particles. The hydrotreater product stream 190 exits hydrotreater 111 and is combined with cracked hydrocarbon stream 210 recycled from hydrocracker 131 to form stream 200. Combined stream 200 is then introduced to fractionator 120 where it is separated into light cut 230, middle cuts 240 and 250, and heavy cut 220. Middle cuts 240 and 250 are preferably a diesel cut and a naphtha cut respectively. It is possible that only one middle distillate cut or more than two middle distillate cuts are desirable as determined by one of ordinary skill in the art based on the desired product composition. Heavy cut 220 is sent to hydrocracker 131 where it is cracked in hydrocracking catalyst zone 130 to cracked hydrocarbon stream 210 which comprises on average lighter hydrocarbons than heavy cut 220. Preferably, but not necessarily, cracked hydrocarbon stream 210 comprises primarily middle distillates and most preferably comprises the most desired middle distillate or middle distillate mix. The cracked hydrocarbon stream 210 is recycled into fractionator 120 for separation. In a preferred embodiment stream 220 (which may comprise primarily C₂₀₊ hydrocarbons) is recycled to extinction.

Referring now to FIG. 2, a reactor scheme similar to FIG. 1 is shown except that stream 210 leaving hydrocracker 131 is not recycled to fractionator 120, but is instead sent to a second fractionator 260.

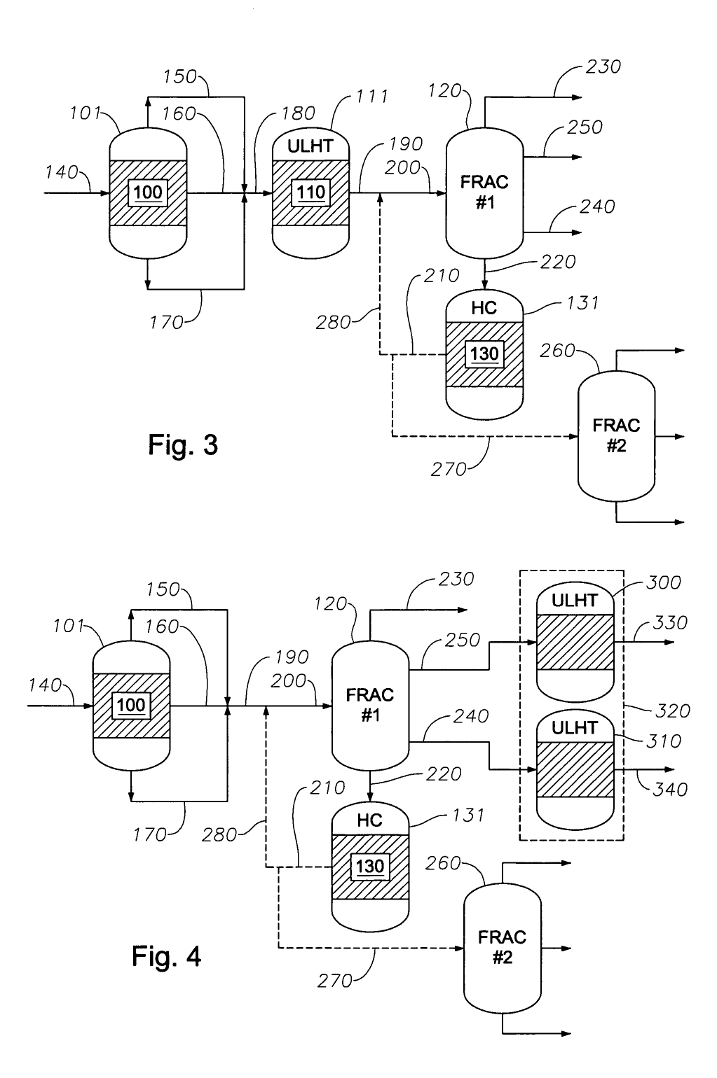

Referring now to FIG. 3, there is shown a reactor scheme similar to that of FIGS. 1 and 2, except that cracked hydrocarbon stream 210 is split into streams 270 and 280. Stream 280 is recycled to fractionator 120 and stream 270 is sent to second fractionator 260. Stream 280 can be combined with stream 190 from the hydrotreater 111 to form stream 200 which is then sent to fractionator 120.

Referring now to FIG. 4, there is shown an embodiment of the present invention including a hydrocarbon synthesis reactor 101 having a hydrocarbon synthesis reaction zone 100, fractionator 120, ultra-low severity hydrotreaters 300 and 310 (which can optionally be one ultra-low severity hydrotreater 320 which alternately hydrotreats individual streams 240 and 250), hydrocracker 131 having hydrocracking reaction zone 130 and second fractionator 260. Feed stream 140 comprising CO and H₂ enter hydrocarbon synthesis reactor 101 where it reacts in reaction zone 100 to form product stream 190. The product stream 190 is combined with recycle stream 280 from hydrocracker 131 to form combined stream 200 being fed into fractionator 120 and separated into streams 230, 240, 250, and 220. Streams 240 and 250 are each fed to ultra-low severity hydrotreaters 310 and 300 respectively where they are hydrotreated and exit ultra-low severity hydrotreaters 310 and 300 as product streams 340 and 330 respectively. Alternatively, individual streams 240 and 250 are fed to a single ultra-low severity hydrotreater 320 alternately. Under this alternate feed configuration, stream 250 is hydrotreated in single ultra-low severity hydrotreater 320 to become product stream 330; then, stream 240 is hydrotreated in single ultra-low severity hydrotreater 320 to become product stream 340. Heavy cut 220 exits fractionator 120 to hydrocracker 131 where it is hydrocracked in hydrocracking zone 130. The hydrocracked stream 210 leaves hydrocracker 131 where it is split into streams 270 and 280. Stream 280 is recycled back to fractionator 120 and stream 270 is sent to second fractionator 260.

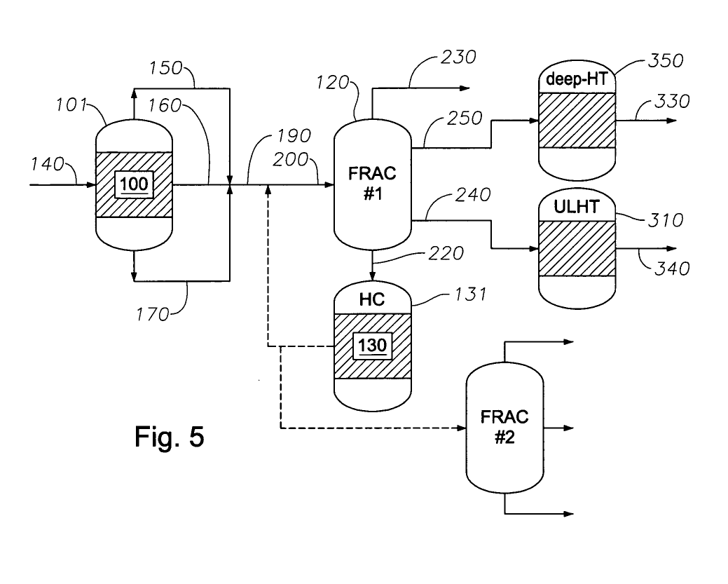

Referring now to FIG. 5, there is shown a configuration similar to FIG. 4 in which the hydrotreaters are downstream of the fractionation. In FIG. 5, one of the downstream hydrotreaters 350 is a traditional or “deep” hydrotreater while the second hydrotreater 310 is a ultra-low severity hydrotreater.

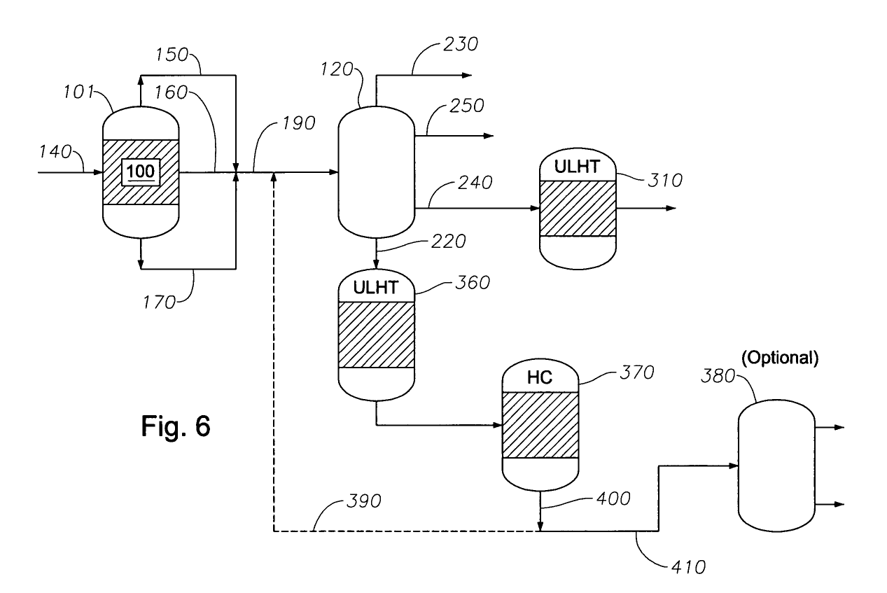

Referring now to FIG. 6, there is shown a configuration comprising a hydrocarbon synthesis reactor 101 having a hydrocarbon synthesis reaction zone 100, a fractionator 120, two ultra-low severity hydrotreaters 310 and 360, hydrocracker 370, and optional second fractionator 380. Feed stream 140 comprising CO and H₂ enter hydrocarbon synthesis reactor 101 where it reacts in reaction zone 100 to form product stream 190. The product stream 190 is combined with recycle stream 390 before being fed into fractionator 120 and separated into streams 230, 240, 250, and 220. Streams 220 and 240 are then hydrotreated in ultra-low severity hydrotreaters 310 and 360 respectively. The stream hydrotreated in ultra-low severity hydrotreater 360 is then sent to hydrocracker 370 where it is cracked into stream 400. Stream 400 can be totally combined with stream 190 to be recycled to fractionator 120, or sent in its entirety to second fractionator 380, or split into streams 390 and 410. If stream 400 is optionally split, at least a portion (i.e., stream 390) is recycled to fractionator 120 and another portion (i.e., stream 410) is sent to second fractionator 380. In alternate embodiments of FIGS. 4-6, stream 220 (which may comprise primarily C₂₀₊ hydrocarbons) is recycled to extinction.

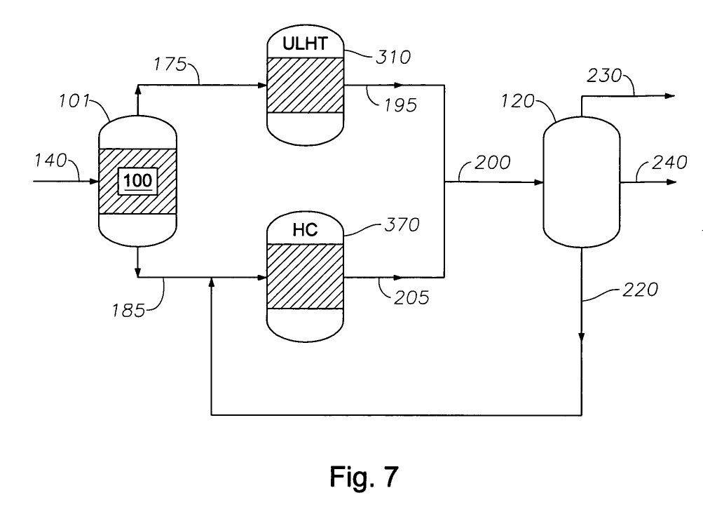

Referring now to FIG. 7, there is shown a configuration comprising a hydrocarbon synthesis reactor 101 having a hydrocarbon synthesis reaction zone 100, a fractionator 120, a ultra-low severity hydrotreater 310, and a hydrocracker 370. Feed stream 140 comprising CO and H₂ enter hydrocarbon synthesis reactor 101 where it reacts in reaction zone 100 to form 2 product streams 175 and 185. Even though FIG. 7 shows that product streams 175 and 185 are represented as two separate streams exiting the hydrocarbon synthesis reactor 101, it is conceivable that one outlet stream is exiting hydrocarbon synthesis reactor 101 for example in a fixed bed reactor embodiment, and this single outlet stream is then divided ex situ (for example by a disengagement step) into the 2 separate product streams 175 and 185. Product stream 175 preferably comprises lighter hydrocarbons than product stream 185. The product stream 175 is hydrotreated in the ultra-low severity hydrotreater 310 to produce hydrotreated stream 195. Product stream 185 is combined with heavy cut stream 220 from fractionator 120 before being fed into hydrocracker 370, where the combined stream is cracked into hydrocracked stream 205. Hydrotreated stream 195 and hydrocracked stream 205 are combined to form stream 200. Stream 200 is then introduced to fractionator 120 where it is separated into light cut 230, middle cut 240, and heavy cut 220. It is to be noted that the combination of streams 195 and 205 is not necessary as long as both streams 195 and 205 are both fed to the fractinator 310. In one embodiment, stream 220 (which may comprise primarily C₂₀₊ hydrocarbons) is recycled to extinction.

The hydrocarbon synthesis reactor 101 preferably comprises a Fischer-Tropsch synthesis and generates primarily hydrocarbons comprising one carbon to 100 carbons or more from a mixture of carbon monoxide (CO) and hydrogen (H₂), also called synthesis gas or syngas. H₂/CO mixtures suitable as a feedstock for conversion to hydrocarbons can be obtained by one or more of the following processes: conversion of biomass, conversion of coal by gasification conversion of light hydrocarbons (such as methane or natural gas) by partial oxidation, reforming or combination thereof. Preferably the hydrogen is provided by free hydrogen, although some Fischer-Tropsch catalysts have sufficient water gas shift activity to convert some water and carbon monoxide to hydrogen and carbon dioxide, for use in the hydrocarbon synthesis process. It is preferred that the molar ratio of hydrogen to carbon monoxide in the feed be greater than 0.5:1 (e.g., from about 0.67 to about 2.5). Preferably, when the hydrocarbon synthesis catalysts comprise cobalt, nickel, and/or ruthenium, the feed gas stream contains hydrogen and carbon monoxide in a molar ratio preferably of about 1.6:1 to about 2.3:1. When the hydrocarbon synthesis catalysts comprise iron, the feed gas stream contains hydrogen and carbon monoxide in a molar ratio preferably between about 1.4:1 and about 2.3:1. The feed gas may also contain carbon dioxide. The feed gas stream should contain only a low concentration of compounds or elements that have a deleterious effect on the catalyst, such as poisons. For example, the feed gas may need to be pretreated to ensure that it contains a low concentration of sulfur or nitrogen compounds such as hydrogen sulfide, hydrogen cyanide, ammonia and carbonyl sulfide. The feed gas is contacted with the catalyst in the reaction zone 100 as shown in FIGS. 1-7. Mechanical arrangements of conventional design may be employed as the reaction zone including, for example, fixed bed, fluidized bed, slurry bubble column or ebullating bed reactors, among others. Accordingly, the preferred size and physical form of the catalyst particles may vary depending on the reactor in which they are to be used. The hydrocarbon synthesis process is typically run in a continuous mode. In this mode, the gas hourly space velocity through the reaction zone typically may range from about 50 to about 10,000 hr¹, preferably from about 300 hr⁻¹ to about 2,000 hr⁻¹. The gas hourly space velocity is defined as the volume of reactants per time per reaction zone volume. The volume of reactant gases is at standard conditions of pressure (101 kPa) and temperature (32° F. or 0° C.). The reaction zone volume is defined by the portion of the reaction vessel volume where reaction takes place and which is occupied by a gaseous phase comprising reactants, products and/or inerts; a liquid phase comprising liquid/waxy products and/or other liquids; and a solid phase comprising catalyst. The reaction zone temperature is typically in the range from about 320° F. to about 570° F. (about 160° C. to about 300° C.). Preferably, the reaction zone is operated at conversion promoting conditions at temperatures from about 375° F. to about 500° F. (about 190° C. to about 260° C.). The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

In accordance with embodiments of the present invention, there is herein a process for producing a predominantly paraffinic stream comprising heteroatomic compounds, said process comprising the following steps: feeding a feedstream comprising synthesis gas to a hydrocarbon synthesis reactor; reacting at least a portion of the feedstream comprising synthesis gas on a hydrocarbon synthesis catalyst to produce a hydrocarbon synthesis product stream; and hydrotreating at least a portion of the hydrocarbon synthesis product stream to produce a hydrotreated stream; wherein the hydrotreated stream comprises no more than an insubstantial amount of olefins; and wherein a substantial amount of the heteroatoms remain attached to their parent molecules during hydrotreating.

### EXAMPLE

A Fischer-Tropsch product was prepared by contacting a synthesis gas mixture (2:1 molar ratio of H₂:CO) with a cobalt catalyst in a continuously stirred tank reactor (CSTR) reactor under typical reaction conditions (430° F. or 221° C.; 350 psia or 2410 kPa). A full range Fischer-Tropsch product was collected and this hydrocarbon stream was fed to a hydrotreater where it was hydrotreated under various conditions. The hydrotreated stream was then distilled to yield a 350-650° F. distillation cut. The hydrotreating catalyst was a commercial nickel based material (NI-3298 E1/16 3F from Engelhard). The hydrotreater comprised a catalytic bed containing about 87 g (100 ml) of said hydrotreating catalyst, and was operated at 350 psia of hydrogen partial pressure in the hydrotreater outlet with a hydrogen flow of 2500 standard cubic feet per barrel of hydrotreater liquid feed (scf/bbl) at a liquid hourly space velocity of 3 hr⁻¹ in trickle flow mode.

In the first experiment the hydrotreater bed temperature was held at 400° F. (204° C.) and in the second experiment at 250° F. (121° C.). The results for Bromine number (ASTM D1149), oxidation stability (ASTM D2274), viscosity (ASTM D445), pour point (ASTM D97), and lubricity HFRR (ASTM D6079) are shown in Table 1. Oxygen content, also included in Table 1, is measured using a Bruker Instruments Avance 400 nuclear magnetic resonance (NMR) spectrometer. The ¹H NMR spectra of the ‘untreated’ sample (i.e., feed of the hydrotreater) and hydrotreated samples were obtained at 400.13 MHz and were run as solutions in deuteriated chloroform (CDCl₃). The signal intensities for olefins, esters and alcohols were compared to those for the total —CH, —CH₂, and —CH₃ groups. The oxygen content in percentage is calculated as —OCH₂ and the percentage approximates weight percentage of O.

TABLE 1

| | Feed | | |
|---|---|---|---|
| | (350-650° F. cut) | Hydrotreating at 250° F. | Hydroeating at 400° F. |
| Bromine number | 5.8 | <0.1 | <0.1 |
| (ASTM D1159) | | | |
| (g Br/100 g) | | | |
| Oxidation Stability | 80 | <0.5 | <0.5 |
| (ASTM D2274) (g/m³) | | | |
| Viscosity @ 40° C. | — | 1.886 | 1.909 |
| (ASTM D445) | | | |
| (cSt) | | | |
| Pour Point | — | 2 | 3 |
| (ASTM D97) | | | |
| (° C.) | | | |
| Oxygenate content (wt % | 0.7 | 0.7 | 0.03 |
| oxygen) | | | |
| Lubricity HFRR(ASTM | — | 340 | 415 |
| D6079) | | | |
| (μm) | | | |

From the results in Table 1 the diesel range stream which has been hydrotreated in an ultra-low severity hydrotreater has an improved lubricity and no decline in oxidation stability (i.e., potential gum formation) as compared to the diesel range stream which has been hydrotreated at 400° F. The Bromine (Br) number method measures the amount of unsaturated hydrocarbons; the example above before hydrotreatment shows a significant presence of unsaturated compounds with a Br number of 5.8 g Br per 100 g of sample; after both hydrotreatments, the Br number was less than 0.1 g Br per 100 g of sample pointing out that both hydrotreating conditions were successful in removing substantially most of the unsaturated compounds so that the hydrotreated samples comprises a significantly reduced amount of olefins. Similarly both hydrotreatment conditions resulted in a much improved oxidation stability (ASTM D2274) of lower than 0.5 g/m³ from a value of 80 g/m³ in the untreated sample. It is conceivable that a lower hydrotreatment temperature (i.e., less than 250° F.) would result in less effective oxidation stability; therefore an oxidation stability value greater than 0.5 g/m³ but smaller than 25 g/m³ is expected at these less severe conditions. Therefore it is highly desirable to have the oxidation stability to be lower than 25 g/m³ in the hydrotreated sample, preferably less than 10 g/m³, more preferably lower than 5 g/m³ and yet more preferably lower than 2 g/m³.

The ultra-low severity hydrotreatment with the nickel based catalyst at 250° F. was successful in retaining most of the oxygenates, as the oxygen content after ultra-low severity treatment resulted in an unchanged value of 0.7 wt %, whereas the hydrotreatment with the nickel based catalyst at 400° F resulted in almost complete removal of the oxygenates with a resulting oxygen content of 0.03 wt %. Typically the 350-650° F. cut of the untreated hydrocarbon synthesis product stream which would feed the hydrotreater would have an oxygen content from about 0.1 wt % to about 15 wt % when the hydrocarbon synthesis reactor uses an iron-based catalyst, and from about 0.1 wt % to about 8 wt % when the hydrocarbon synthesis reactor uses a cobalt-based catalyst. It is conceivable that a higher hydrotreatment temperature (i.e., greater than about 250° F. and less than about 350° F.) would result in a more effective removal of oxygen atoms; therefore the oxygen content of a stream comprising primarily a 350-650° F. boiling range after an ultra-low severity hydrotreating could be lower than the untreated stream comprising primarily a 350-650° F. boiling range. It is preferable therefore to retain as much as of the oxygen content as possible, retaining at least 50% of the oxygen content, preferably at least 75%, and more preferably at least 90%, to obtain the desirable oxygen content equal to or greater than 0.1 wt %, for improving diesel or middle distillate properties after the ultra-low severity hydrotreating.

Therefore a hydrotreated middle distillate cut derived from synthesis gas, obtained after an ultra-low severity hydrotreating step has preferably an oxidation stability (gum) less than 25 g/m³; and an oxygen content equal to or greater than 0.1 wt %. Similarly, a hydrotreated diesel product derived from synthesis gas and obtained after an ultra-low severity hydrotreating without the addition of property enhancing agents has preferably the following properties:

Bromine number <0.1 gBr/100 g;

Oxidation stability (gum) ≦25 g/m³;

oxygen content ≧0.1 wt %; and

lubricity HFRR ≦400 μm.

As used herein, to “hydroprocess” means to treat a hydrocarbon stream with hydrogen.

“Hydrocarbon synthesis” can be any method now known or later discovered for synthesizing liquid hydrocarbons. An example is the Fischer-Tropsch process.

To “hydrotreat” means to treat a hydrocarbon stream with hydrogen without making any substantial change to the carbon backbone of the molecules in the hydrocarbon stream. For example, hydrotreating a hydrocarbon stream comprising predominantly H₂C═CH—CH₂—CH₂—CH₃ would yield a hydrocarbon stream comprising predominantly CH₃—CH₂—CH₂—CH₂—CH₃. As used herein, to “hydrocrack” means to split an organic molecule and add hydrogen to the resulting molecular fragments to form two smaller hydrocarbons (e.g., C₁₀H₂₂+H₂→C₄H₁₀ and skeletal isomers+C₆H₁₄ and skeletal isomers). Because a hydrocracking catalyst can be active in hydroisomerization, there can be some skeletal isomerization during the hydrocracking step, therefore isomers of the smaller hydrocarbons can be formed. Methods for hydrocracking are legion and well known in the art. Preferably, the hydrocracking takes place over a platinum catalyst at a temperature of about 550° F. to about 750° F. (260-400° C.) and at a pressure of about 500 psig to about 1500 psig (3,550-10,440 kPa).

“Heteroatomic compounds” are organic compounds which comprise not only carbon and hydrogen, but also other atoms, such as nitrogen, sulfur, oxygen. The non-carbon and non-hydrogen atoms (e.g., oxygen, sulfur and nitrogen, respectively) are “heteroatoms”. Examples of heteroatomic compounds comprising oxygen are alcohols, aldehydes or ketones. Examples of heteroatomic compounds comprising nitrogen are amines. For example, acetone (CH₃COCH₃) and dipropyl amine ((C₃H₇)₂NH) are heteroatomic compounds. With respect to, for example, acetone, a related heteroatomic compound is isopropyl alcohol ((CH₃)₂CHOH). In a situation in which acetone is converted to isopropyl alcohol, the heteroatom (oxygen), although differently bonded, remains attached to its parent molecule (e.g., is not removed from its carbon backbone). Likewise, when, for example, acetone has gone through a process unconverted, the heteroatom (oxygen) has also remained attached to its parent molecule.

As used herein, “ultra-low severity hydrotreatment” means hydrotreatment at conditions such that a substantial portion of the olefins in a stream becomes saturated, but a substantial amount of the heteroatoms in the stream remain attached to their parent molecule. The two most important factors in determining whether a hydrotreating process does not convert a substantial amount of oxygenates to paraffins are catalyst composition and temperature. Ultra-low severity hydrotreating can take place with hydrotreating catalysts comprising at least one of the following metals: a group VIB metal (from the previous IUPAC notation), such as molybdenum (Mo) and tungsten (W), or a group VIII metal, such as nickel (Ni), palladium (Pd), platinum (Pt), ruthenium (Ru), iron (Fe), cobalt (Co). Highly active catalysts, such as those comprising Ni, Pd, Pt, W, Mo, Ru or combinations thereof, must be operated at relatively low temperatures between about 180° F. and about 350° F. (about 80-180° C.), more preferably between about 180° F. and about 320° F. (about 80-160° C.), still more preferably between about 180° F. to about 300° F. (about 80-150° C.). By way of example only, a highly active catalyst such as a nickel-based catalyst begins to convert a substantial amount of oxygenates at about 220° F. In contrast, a less active catalysts such as those comprising Fe or Co do not begin to convert a substantial amount of the oxygenates until it reaches a temperature of about 350° F. For these catalysts with lower hydrotreating activity (e.g., Co or Fe), a preferred temperature range for ultra-low severity hydrotreating is between about 350° F. and about 570° F. (about 180-300° C). Additionally, there are other parameters such as for example, pressure and liquid hourly space velocity which may be varied by one of ordinary skill in the art to effect the desired ultra-low severity hydrotreating. Preferably the hydrogen partial pressure is between about 100 psia and about 1,000 psia (690-6900 kPa), more preferably between about 300 psia and about 500 psia (2060-3450 kPa). The liquid hourly space velocity is preferably between 1 and 10 hr⁻¹, more preferably between 0.5 and 6 hr⁻¹, still more preferably between about 1 and about 5 hr⁻¹. It should be understood that the hydrotreating catalyst for ultra-low severity hydrotreatment can be with or without support, and can comprise promoters to improve catalyst performance and/or support structural integrity.

As used herein, a “diesel” is any hydrocarbon cut having at least a portion which falls within the diesel range. The diesel range in this application includes hydrocarbons which boil in the range of about 300° F. to about 750° F. (about 150-400° C.), preferably in the range of about 350° F. to about 650° F. (about 170-350° C.).

As used herein, a “middle distillate” means a hydrocarbon stream which includes kerosene, home heating oil, range oil, stove oil, and diesel that has a 50 percent boiling point in the ASTM D86 standard distillation test falling between 371° F. and 700° F.

As used herein, “deep hydrotreatment” means hydrotreatment over a hydrotreating catalyst comprising at least one metal from the group consisting of Ni, Pd, Pt, Mo, W, and Ru, preferably comprising Ni, over at temperatures above 350° F. (170° C.), preferably from 350° F. to about 600° F. (315° C.), more preferably from 360° F. to about 600° F. (180-315° C.), with a hydrogen partial pressure in the hydrotreater outlet between about 100 psia and about 2,000 psia (690-13,800 kPa).

Should the disclosure of any of the patents and publications that are incorporated herein by reference conflict with the present specification to the extent that it might render a term unclear, the present specification shall take precedence.

While the preferred embodiments of the invention have be disclosed herein, it will be understood that various modifications can be made to the system described herein without departing from the scope of the invention. Without further elaboration, it is believed that one skilled in the art can, using the description herein, utilize the present invention to its fullest extent.

What is claimed is:

1. A method for processing at least a portion of a hydrocarbon synthesis product stream comprising paraffins, olefins, and at least one heteroatomic compound, the method comprising:
hydrotreating at least a portion of the hydrocarbon synthesis product stream in a hydrotreater to produce a hydrotreated stream;
wherein the hydrotreated stream comprises an insubstantial amount of olefins; and
wherein a substantial amount of the heteroatoms remain attached to their parent molecules during hydrotreating.

2. The method of claim 1 wherein the hydrocarbon synthesis product stream comprises a Fischer-Tropsch product stream.

3. The method of claim 1 wherein at least one heteroatomic compound is an oxygenate.

4. The method of claim 1 wherein at least a portion of a hydrocarbon synthesis product stream is fractionated upstream of the hydrotreater.

5. The method of claim 4 wherein the at least a portion of the hydrocarbon synthesis product stream comprises primarily diesel.

6. The method of claim 1 wherein the hydrotreater comprises a catalyst, wherein the catalyst comprises at least one metal selected from the group consisting of Ni, Pd, Pt, Ru, Mo, W, Fe, and Co.

7. The method of claim 6 wherein the hydrotreater comprises a catalyst, and said catalyst comprises at least one metal selected from the group consisting of Ni, Pd, Pt, Ru, Mo, and W.

8. The method of claim 6 wherein the hydrotreater comprises a catalyst comprising Ni.

9. The method of claim 8 wherein the hydrotreater is operated at a temperature of between about 180° F. and about 350° F.

10. The method of claim 8 wherein the hydrotreater is operated at a temperature of between about 180° F. and about 325° F.

11. The method of claim 8 wherein the hydrotreater is operated at a temperature of between about 180° F. and about 300° F.

12. The method of claim 6 wherein the catalyst comprises Co, Fe, or combination thereof.

13. The method of claim 12 wherein the hydrotreater is operated at a temperature of between about 350° F. and about 570° F.

14. The method of claim 1 further comprising hydrocracking at least a portion of the hydrotreated stream to produce a hydrocracked stream.

15. The method of claim 1 wherein the hydrotreated stream comprises a diesel fraction and wherein the diesel fraction has an oxygen content of about 0.1 wt % or greater and an oxidation stability of about 25 g/m³ or less.

16. The method of claim 1 wherein the hydrotreater has a liquid hourly space velocity between about 0.1 to about 10 hr⁻¹.

17. The method of claim 1 wherein the hydrotreater has a liquid hourly space velocity between about 0.5 to about 6 hr⁻¹.

18. A hydroprocessed product stream wherein the hydroprocessed product stream is produced by the method comprising:
hydrotreating at least a portion of a hydrocarbon synthesis product stream to produce a hydroprocessed product stream;
wherein at least a portion of a hydrocarbon synthesis product stream comprises paraffins, olefins, and at least one heteroatomic compound:
wherein the hydroprocessed product stream comprises no more than an insubstantial amount of olefins; and
wherein a substantial amount of the heteroatoms remain attached to their parent molecules during hydrotreating.

19. The hydroprocessed product stream of claim 18 wherein the hydrocarbon synthesis product stream comprises a Fischer-Tropsch product stream.

20. The product stream of claim 18 wherein at least one heteroatomic compound is an oxygenate.

21. The product stream of claim 18 wherein the at least a portion of a hydrocarbon synthesis product stream is fractionated upstream of the hydrotreater.

22. The product stream of claim 21 wherein the at least a portion of the hydrocarbon synthesis product stream comprises primarily diesel.

23. The method of claim 18 wherein the hydrotreater comprises a catalyst, wherein the catalyst comprises at least one metal selected from the group consisting of Ni, Pd, Pt, Ru, Mo, W, Fe, and Co.

24. The method of claim 23 wherein the hydrotreater comprises a catalyst, and said catalyst comprises at least one metal selected from the group consisting of Ni, Pd, Pt, Ru, Mo, and W.

25. The product stream of claim 24 wherein the hydrotreater comprises a nickel catalyst.

26. The product stream of claim 25 wherein the hydrotreater is operated at a temperature of between about 180° F. and about 350° F.

27. The product stream of claim 26 wherein the hydrotreater is operated at a temperature of between about 180° F. and about 300° F.

28. The method of claim 23 wherein the catalyst comprises Co, Fe, or combination thereof.

29. The method of claim 28 wherein the hydrotreater is operated at a temperature of between about 350° F. and about 570° F.

30. The product stream of claim 18 wherein the hydroprocessed product stream comprises a diesel fraction and wherein the diesel fraction has an oxygen content of about 0.1 wt % or greater and an oxidation stability of about 25 g/m³ or less.

31. A process for producing a predominantly paraffinic stream comprising heteroatomic compounds, the process comprising:
feeding a feedstream comprising synthesis gas to a hydrocarbon synthesis reactor;
reacting at least a portion of the feedstream comprising synthesis gas on a hydrocarbon synthesis catalyst to produce a hydrocarbon synthesis product stream;
hydrotreating at least a portion of the hydrocarbon synthesis product stream to produce a hydrotreated stream;
wherein the hydrotreated stream comprises no more than an insubstantial amount of olefins; and
wherein a substantial amount of the heteroatoms remain attached to their parent molecules during hydrotreating.

32. The process of claim 31 wherein the hydrocarbon synthesis product stream is a Fischer-Tropsch product stream.

33. The process of claim 31 wherein at least one heteroatomic compound is an oxygenate.

34. The process of claim 31 wherein the hydrocarbon synthesis product stream is fractionated upstream of the hydrotreater.

35. The process of claim 34 wherein the at least a portion of the hydrocarbon synthesis product stream comprises primarily diesel.

36. The process of claim 31 wherein the hydrotreater comprises a hydrotreating catalyst, said hydrotreating catalyst comprising at least one metal selected from the group consisting of Ni, Pd, Pt, Ru, Mo, W, Fe, and Co.

37. The process of claim 36 wherein the hydrotreating catalyst comprises at least one metal selected from the group consisting of Ni, Pd, Pt, Ru, Mo, and W.

38. The process of claim 37 wherein the hydrotreating catalyst comprises Ni.

39. The process of claim 38 wherein the hydrotreater is operated at a temperature of between about 180° F. and about 350° F.

40. The process of claim 39 wherein the hydrotreater is operated at a temperature of between about 180° F. and about 300° F.

41. The product stream of claim 36 wherein the hydrotreating catalyst comprises Co, Fe, or combination thereof.

42. The process of claim 41 wherein the hydrotreater is operated at a temperature of between about 350° F. and about 570° F.

43. The process of claim 31 wherein the hydrotreated stream comprises a diesel fraction and wherein the diesel fraction has an oxygen content of about 0.1 wt % or greater and an oxidation stability of about 25 g/m³ or less.

44. A hydrotreated diesel product derived from synthesis gas having the following properties:
Oxidation stability ≦25 g/m³;
oxygen content ≧0.1 wt %; and
lubricity HFRR ≦400 μm;
wherein the hydrotreated diesel product consists essentially of molecules which have been hydrotreated.

45. A process for producing middle distillates from synthesis gas, the process comprising:
A) reacting a feedstream comprising synthesis gas over a hydrocarbon synthesis catalyst at conditions effective to convert the synthesis gas to a first hydrocarbon stream comprising paraffins, olefins, and heteroatomic compounds;
B) hydrotreating the first hydrocarbon stream in a first hydrotreater to produce a second hydrocarbon stream characterized by a significantly reduced amount of olefins while a substantial amount of said heteroatomic compounds have the heteroatoms attached to their parent molecules;
C) fractionating the second hydrocarbon stream in a first fractionator to produce at least a heavy hydrocarbon cut and at least a first middle distillate cut;
D) hydrocracking the heavy hydrocarbon cut from said fractionating step C) to form a third hydrocarbon stream; and
E) fractionating the third hydrocarbon stream to produce at least a second middle distillate cut.

46. The process of claim 45 wherein step E) and step C) take place in the same fractionator.

47. The process of claim 45 wherein step E) is performed in a fractionator different than the first fractionator of step C).

48. The process of claim 45 wherein step E) comprises fractionating a portion of said third hydrocarbon stream in the first fractionator of step C) and fractionating another portion of said third hydrocarbon stream in a second fractionator.

49. The process of claim 45 wherein the hydrocarbon synthesis catalyst comprises a group VIII metal.

50. The process of claim 45 wherein the hydrocarbon synthesis catalyst comprises cobalt or iron.

51. The process of claim 45 wherein the first hydrotreater comprises a hydrotreating catalyst, and the hydrotreating catalyst comprises at least one metal selected from the group consisting of Ni, Pd, Pt, Ru, Mo, W, Fe, and Co.

52. The process of claim 51 wherein the first hydrotreater comprises a hydrotreating catalyst, and the hydrotreating catalyst comprises at least one metal selected from the group consisting of Ni, Pd, Pt, Ru, Mo, and W.

53. The process of claim 52 wherein the hydrotreating catalyst comprises Ni.

54. The process of claim 53 wherein the first hydrotreater is operated at a temperature of between about 180° F. and about 350° F.

55. The process of claim 53 wherein the first hydrotreater is operated at a temperature of between about 180° F. and about 300° F.

56. The process of claim 51 wherein the hydrotreating catalyst comprises Co, Fe, or combination thereof.

57. The process of claim 45 wherein the first hydrotreater is operated at a temperature of between about 350° F. and about 570° F.

58. The process of claim 45 wherein at least a portion of the heteroatomic compounds formed in step A) contain oxygen.

59. The process of claim 58 wherein the hydrotreated second hydrocarbon stream comprises primarily a 350°-650° F. boiling range fraction, and said fraction has an oxygen content of about 0.1 wt % or greater and an oxidation stability of about 25 g/m³ or less.

60. The process of claim 58 wherein the first middle distillate stream has an oxygen content of about 0.1 wt % or greater, and an oxidation stability of about 25 g/m³ or less.

61. A process for producing middle distillates from synthesis gas, the process comprising:
A) reacting a feedstream comprising synthesis gas over a hydrocarbon synthesis catalyst at conditions effective to convert the synthesis gas to a first hydrocarbon stream comprising paraffins, olefins, and heteroatomic compounds;
B) fractionating the first hydrocarbon stream in a first fractionator to produce a light hydrocarbon stream, a heavy hydrocarbon stream, and a first middle distillate stream;
C) hydrocracking the heavy hydrocarbon stream from step B) to form a third hydrocarbon stream;
D) fractionating the hydrocracked third hydrocarbon stream from step C) to produce at least a second middle distillate stream; and
E) hydrotreating the first middle distillate stream from step B) in a first hydrotreater to produce a hydroteated middle distillate stream characterized by a significantly reduced amount of olefins while a substantial amount of said heteroatomic compounds formed in step A) have the heteroatoms attached to their parent molecules.

62. The process of claim 61 wherein step D) comprises fractionating said third hydrocarbon stream in the first fractionator of step B).

63. The process of claim 61 wherein step D) comprises fractionating said third hydrocarbon stream in a second fractionator.

64. The process of claim 61 wherein step D) comprises fractionating a portion of said third hydrocarbon stream in the first fractionator of step B) and fractionating another portion of said third hydrocarbon stream in a second fractionator.

65. The process of claim 61 wherein the hydrocarbon synthesis catalyst comprises a group VIII metal.

66. The process of claim 61 wherein the hydrocarbon synthesis catalyst comprises cobalt or iron

67. The process of claim 61 wherein the first hydrotreater comprises a hydrotreating catalyst, and said hydrotreating catalyst comprises at least one metal selected from the group consisting of Ni, Pd, Pt, Ru, Mo, W, Fe, and Co.

68. The process of claim 67 wherein the first hydrotreater comprises a hydrotreating catalyst, and said hydrotreating catalyst comprises at least one metal selected from the group consisting of Ni, Pd, Pt, Ru, Mo, and W.

69. The process of claim 68 wherein the hydrotreating catalyst comprises Ni.

70. The process of claim 69 wherein the first hydrotreater is operated at a temperature of between about 180° F. and about 350° F.

71. The method of claim 67 wherein the catalyst comprises Co, Fe, or combination thereof.

72. The method of claim 71 wherein the hydrotreater is operated at a temperature of between about 350° F. and about 570° F.

73. The process of claim 61 wherein at least a portion of the heteroatomic compounds formed in step A) contain oxygen.

74. The process of claim 73 wherein the first middle distillate stream from step C) contains diesel, the diesel comprising primarily a 350°-650° F. boiling range fraction and having an oxygen content of about 0.1 wt % or greater and an oxidation stability of about 25 g/m³ or less.

75. The process of claim 61 further comprising:
F) hydrotreating at least a portion of the light hydrocarbon stream from step B) in a second hydrotreater to produce a hydrotreated light hydrocarbon stream comprising naphtha.

76. The process of claim 75 wherein hydrotreating in step F) is performed over a nickel-based catalyst at a temperature above 350° F.

77. The process of claim 75 wherein hydrotreating in step F) is performed over a nickel-based catalyst at a temperature between about 180° F. and 350° F., wherein the hydrotreated light hydrocarbon stream is characterized by a significantly reduced amount of olefins while a substantial amount of said heteroatomic compounds have the heteroatoms attached to their parent molecules.

78. The process of claim 75 wherein the first hydrotreater and the second hydrotreater are the same hydrotreater, and steps E) and step F) are performed alternately.

79. A method for processing at least a portion of a hydrocarbon synthesis product stream comprising paraffins, olefins, and at least one heteroatomic compound, the method comprising:
hydrotreating at least a portion of the hydrocarbon synthesis product stream in an ultra-low severity hydrotreater.
